(19) United States
(12) Patent Application Publication
(10) Pub. No.: US 2008/0032404 A1
(43) Pub. Date: Feb. 7, 2008

(54) METHOD FOR CONSTRUCTING EUKARYOTIC CELLS HAVING MULTIPLE GENOMIC DELETIONS AND/OR INSERTIONS

(75) Inventors: Frederick P. Roth, Newton, MA (US); Oliver D. King, Somerville, MA (US); Lan Zhang, San Francisco, CA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/780,132

(22) Filed: Jul. 19, 2007

Related U.S. Application Data
(63) Continuation of application No. PCT/US2006/003626, filed on Feb. 3, 2006.
(60) Provisional application No. 60/649,868, filed on Feb. 3, 2005.

(51) Int. Cl. C12N 15/87

(52) U.S. Cl. 435/440

(57) ABSTRACT

The present invention provides methods for engineering multiple genomic deletions and/or insertions in eukaryotic cells.

### RELATED APPLICATIONS

This application is a continuation of International patent application number PCT/US2006/003626, filed Feb. 3, 2006; which claims priority from U.S. provisional application No. 60/649,868, filed Feb. 3, 2005; each of which is hereby incorporated herein by reference in its entirety for all purposes.

### FIELD

Embodiments of the present invention relate to the production of eukaryotic cells having multiple genomic deletions and/or insertions.

### BACKGROUND

Eukaryotic cells having many gene alterations, i.e., deletions and/or insertions, can be extremely useful research reagents. For example, cells missing many efflux pumps (Decottignies et al. (1998) J. Biol. Chem. 273:12612; Nakamura et al. (2001) Antimicrob. Agents. Chemother. 45:3366) can facilitate study of drug mechanism of action (see, e.g., Miyamoto et al. (2002) J. Biol. Chem. 277:28810) or may be useful in cloning heterologous efflux pump genes. Deletion of twenty genes by a successive deletion procedure has resulted in a yeast strain deficient in glucose transport, which proved useful both in subsequent “add-back” experiments and in cloning of heterologous hexose transporters (Wieczorke et al. (1999) FEBS Lett. 464:123; Wieczorke et al. (2003) Cell Physiol. Biochem., 13:123). A human protein pathway can be studied by “transplanting” all of the required genes to a more tractable genetic system such as S. cerevisiae. To study these exogenous genes in yeast, one must often eliminate the orthologous yeast genes.

Current methods of constructing eukaryotic cells having multiple genomic deletions and/or insertions are associated with many difficulties. Sequential gene replacement, for example, is laborious and there are too few selectable markers to delete dozens of genes without eliminating and re-using markers. Systems have been developed for the purpose of eliminating markers after their use in a gene replacement so that they may be re-used as markers. For example, a Cre-Lox system has been used (Delneri et al. (2000) Gene, 252:127). In this approach, a deletion is introduced using a selectable marker flanked by LoxP sites, the selectable marker is excised by inducing Cre, and this process is repeated with subsequent markers. Unfortunately, in strains with multiple deletions, non-adjacent LoxP sites can recombine causing widespread chromosomal arrangements (Wieczorke et al. (1999) FEBS Lett., 464:123; Wieczorke et al. (2003) Cell Physiol. Biochem., 13:123).

Another problem with constructing eukaryotic cells having multiple deletions and/or insertions occurs when there are synthetic sick or lethal pairs among genes to be deleted (i.e., gene pairs such that the fitness of cells with both genes deleted is less than the deletion of each of the genes singly), which may lead to different “dead ends” (i.e., strains such that the next desired deletion leads to a cell with unacceptably low fitness) depending on the order in which deletions are introduced. Determining the order in which deletions should be made so as to permit the maximal number of desired deletions to be incorporated into a genome requires sequential trial and error, and can be impracticable using methods currently known in the art. Although random mutagenesis may be used to generate a large number of mutations to disrupt many genes, characterizing the genotype of the resulting strain requires re-sequencing of the entire genome. Such a method is undesirable, in that genes other than those of interest are often also affected. Loss, gain or other alteration(s) of function(s) of genes beyond those whose disruption is desired can produce phenotypic effects that may, in combination with one or more desired mutations, confound subsequent attempts at analysis of cells or organisms bearing deletions. Such alterations may otherwise defeat the purpose of the intended deletions, such as by interfering with the production or suppression of production of a polypeptide, protein, biomolecule, whose production or lack thereof by the deletion-bearing cell or organism is sought, or even by killing the cell or organism. Furthermore, a null allele (i.e., leading to an absent or effectively absent a gene product), which is typically more desirable than a point mutation or a partial deletion, will not be the predominant result of such mutagenesis.

There is also a need for improved methods of generating S. cerevisiae strains carrying multiple insertions, e.g., to study the function of a collection of human genes in a more genetically tractable model system, or to engineer S. cerevisiae to produce antibiotics requiring multiple biosynthetic genes. Similar technological limitations currently apply to the production of multiple insertion strains as described for multiple deletion strains: 1) a shortage of independently selectable markers, 2) genomic rearrangements and “dead ends” in the sequential use, elimination, and re-use of selectable markers, and 3) unintended effects caused by random insertional mutagenesis, e.g., using transposons carrying the desired insertions.

Accordingly, there is a need for eukaryotic cells and methods of generating eukaryotic cells having multiple deletions and/or insertions. Eukaryotic cells having many gene deletions and/or insertions would permit rapid discovery of genetic interactions, allowing efficient discovery of synthetic lethality between gene pairs, as well as efficient discovery of higher-order genetic interaction involving three or more genes. Methods for producing such cells also would provide the tools to determine a “minimal eukaryotic genome”. Furthermore, sets of compatible deletions could be enriched for genes within the same pathway, allowing identification of pathways and new pathway members. Finally, the ability to generate strains carrying multiple precise insertions would facilitate the study of exogenous genes in a model organism with more tractable genetic properties.

### SUMMARY

Methods of generating multiple deletions and/or insertions in eukaryotic organisms known in the art suffer from a variety of limitations including a need for laborious experimentation, and a lack of suitable markers for screening. The present invention overcomes such limitations and provides methods for engineering multiple genomic deletions and/or insertions in eukaryotic cells.

It is an object of the present invention to provide methods for engineering multiple genomic deletions and/or insertions in eukaryotic cells. These methods reduce the amount of labor needed and reduce unintended mutagenesis compared with mutagenic methods known in the art, and allow for selection of cells expressing multiple desired genomic alterations using only one or a small number of identifiable markers.

It is a further object of the present invention to provide methods for engineering eukaryotic cells and organisms having large numbers of genomic deletions and/or insertions.

The present invention is based in part on the discovery of a method of deleting and/or inserting nucleic acids from a first nucleic acid sequence of interest (e.g., a first endogenous gene) in a first eukaryotic cell and inserting an identifiable marker, deleting and/or inserting nucleic acids from a second nucleic acid sequence of interest (e.g., a second endogenous gene) in a second eukaryotic cell and inserting an identifiable marker. The first and second cells are combined and homologous recombination is allowed to occur. This generates a cell having deleted and/or inserted nucleic acid sequences in first and second nucleic acid sequences of interest associated with identifiable markers. The presence of the identifiable markers is screened for, and the process is repeated to obtain a eukaryotic cell having multiple genomic deletions and/or insertions in a variety of nucleic acid sequences (e.g., genes) of interest.

In certain aspects, a cell having a deleted and/or inserted nucleic acid sequence of interest associated with an identifiable marker may have one or more additional nucleic acid sequences of interest deleted and/or inserted and associated with identifiable markers using a variety of techniques for introducing an exogenous nucleic acid sequence into a target cell. Such techniques include, but are not limited to, calcium phosphate or calcium chloride co-precipitation, DEAE-dextran-mediated transfection, lipofection, electroporation, optoporation, injection and the like, which are discussed further herein.

Accordingly, embodiments of the present invention are directed to methods of altering multiple nucleic acid sequences of interest (e.g., endogenous genes) in a target eukaryotic cell. In one aspect, methods of altering multiple nucleic acid sequences of interest include replacing the nucleic acid sequences of interest with an identifiable marker. In certain aspects, a single identifiable marker (i.e., a continuously selectable marker) is used to replace each nucleic acid sequence of interest. In other aspects, two or more different identifiable markers are used to replace nucleic acid sequences of interest. In certain aspects, identifiable markers include markers that may be detected visually, such as by fluorescence, luminescence, enzyme systems, and the like. Expression of the identifiable markers can be regulated by an inducible promoter.

Detection of identifiable markers may be performed by direct or indirect (e.g., using a microscope, camera or other device) visual inspection or using a detection means such as a fluorescence-detecting device or scanner, photometric device (e.g., a spectrophotometer), mass spectrometer, signal-activated cell sorting device (e.g., a fluorescence-activated cell sorter, or FACS) or other fluidics device. If multiple identifiable markers (i.e., those that produce different signals) are to be used, the detection means selected should be able to detect each type of signal generated by the various identifiable markers and differentiate among them. If a single identifiable marker instead is to be used, increases in marker signal intensity should be detectable by the detection means in a manner that correlates well with marker gene copy number in the marker-bearing cell or organism. To that end, the signal detection means for a given identifiable marker is most usefully selected so as to maximize the number of marker gene copies that it can detect before the relationship between signal intensity and marker gene copy number becomes non-linear.

According to certain embodiments of the present invention, a first nucleic acid sequence in first target cell is replaced with an identifiable marker and a second nucleic acid sequence in second target cell is replaced with an identifiable marker. The two cells are then combined and genetic material from the first and second cells is allowed to undergo homologous recombination such that nucleic acid sequences from the first cell and the second cell are exchanged. In certain aspects, the first and second cells are combined by mating. After recombination, a single cell having both the first and second alteration is selected. In certain aspects of the invention, the cell having both the first and second alteration is selected by detecting an increase in identifiable marker expression when compared to a cell having only one alteration. Cells are combined and selection of cells having increased identifiable marker expression is repeated until a desired number of replacements of a desired number of nucleic acid sequences with an identifiable marker is achieved. In certain aspects, a cell having a first nucleic acid replaced with an identifiable marker has one or more additional replacements generated by introducing a nucleic acid sequence into the cell using any of the molecular biological techniques described herein (e.g., transfection, transformation and the like).

According to other embodiments of the present invention, cells deficient in one or more proteins are provided. In certain aspects, cells deficient in some proteins, a majority of proteins or all proteins involved in one or more particular cellular pathways are provided. In certain aspects, cells are provided in which some endogenous proteins, a majority of endogenous proteins or all endogenous proteins involved in one or more particular cellular pathways are replaced with exogenous proteins. In still other aspects, a minimal genome is created.

Embodiments of the present invention have particular applications as tools for molecular biology and biochemistry. In particular, the present invention provides cells and organisms that are useful for studying gene function, protein structure and function, and developmental biology, characterizing biological pathways, and creating genetically modified organisms (e.g., transgenic, knockout and the like). Such cells and organisms also are useful in the manufacture of proteins and other biomolecules (whether or not endogenous to the particular cell or organism), such as to increase yield over that which could be achieved in the cell or organism in the absence of engineering according to the present invention. Such cells and organisms are also useful to enable the production of proteins or other biomolecules that the cells or organisms ordinarily could not produce. The invention further provides cells useful for cell-based therapies (e.g., for transplantation into a recipient mammal, including a human) and vaccines for human and veterinary use.

Embodiments of the present invention also have particular application in generating cells having multiple genomic deletions that could not previously be made due to the lethality of the deletions when combined using traditional methods. The present invention enables the elucidation of synthetic lethality between gene pairs, as well as the elucidation of higher order genetic interactions involving three or more genes. The present invention is also useful for creating minimal eukaryotic genomes. The present invention is also useful for enriching for genes within one or more particular cellular pathways, thus enabling the elucidation of the functional relationship of individual genes in the particular pathways, whether of the cell or organism engineered according to the present invention or of a second cell or organism, whose genes are introduced into such an engineered cell or organism, as well as enabling the identification of novel cellular pathways.

### DETAILED DESCRIPTION

The present invention provides a novel method of deleting and/or inserting nucleic acids from a first nucleic acid sequence of interest (e.g., a first endogenous gene) in a first eukaryotic cell and inserting an identifiable marker and deleting and/or inserting nucleic acids from a second nucleic acid sequence of interest (e.g., a second endogenous gene) in a second eukaryotic cell and inserting an identifiable marker. The first and second cells are combined and homologous recombination is allowed to occur. This will generate a cell having deleted and/or inserted nucleic acid sequences in first and second nucleic acid sequences of interest associated with identifiable markers. The presence of identifiable markers is screened for and the process is repeated to obtain a eukaryotic cell or organism having multiple genomic deletions and/or insertions in a variety of nucleic acid sequences (e.g., genes) of interest. In certain aspects of the invention, a cell having a deleted and/or inserted nucleic acid sequence of interest associated with an identifiable marker may have one or more additional nucleic acid sequences of interest deleted and/or inserted and associated with identifiable markers using a variety of techniques for introducing an exogenous nucleic acid sequence into a target cell.

In certain aspects of the invention, the same identifiable marker (i.e., a continuously selectable marker) is associated with a nucleic acid sequence of interest in both the first and second eukaryotic cells. The presence of the same identifiable marker (i.e., continuously selectable marker) associated with both nucleic acid sequences of interest (i.e., after homologous recombination) is determined by detecting an increase in an activity of the specific identifiable marker. An additive increase in marker activity will likewise be observed for each addition of the same identifiable marker (i.e., continuously selectable marker) to create a cell having multiple mutations and/or deletions.

The terms “identifiable marker” and “continuously selectable marker” as used herein include, but are not limited to, observable markers that used to replace or are otherwise genetically linked to a desired allele (e.g. insertion or deletion) of a gene of interest. Identifiable markers and continuously selectable marker include, without limitation, visually detectable markers that may be positively and/or negatively selected for and/or screened for using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucorinidases, phosphatases, peroxidases, cholinesterases and the like. Identifiable or continuously selectable markers within the scope of the invention produce a phenotype that distinguishes strains carrying more genetic copies of the marker from strains carrying no copies or fewer copies of the marker.

Identifiable markers also include, but are not limited to, drug sensitivity genes wherein the gene product imparts a certain sensitivity to the cell or organism that affects the ability of the cell or organism to tolerate a drug in the growth medium. For example, the drug G418 can be used to select for the kanr drug resistance gene in many organisms. The gene dosage, i.e., the number of copies of the drug resistance gene, has been shown in the case of kanr and in many others to correlate with the degree of drug resistance conferred (Scorer et al. (1994) Biotechnology 12:181; Yasui et al. (2004) Cancer Res. 64:1403, incorporated herein by reference in their entirety for all purposes). Methods of screening identifiable markers are known in the art, and kits and equipment for screening identifiable markers are commercially available.

In certain aspects of the invention, an identifiable marker is operatively linked to one or more regulatory sequences, such that expression of the identifiable marker may be controlled (e.g., up-regulated or down-regulated). The term “operably linked” is intended to mean that the identifiable marker is linked to the regulatory sequence(s) in a manner which allows for expression of the nucleotide sequence encoding the identifiable marker in a target cell when that sequence is introduced into the target cell, whether such sequence is present on chromosomal DNA of another cell to which the target cell is exposed, resulting in mating or other cell-to-cell transfer of DNA, or whether it is instead present on a nucleic acid molecule, including without limitation a nucleic acid vector, which molecule is introduced into the target cell by transfection, transformation or other means known in the art.

In certain aspects of the invention, a reference marker is introduced, such that the phenotype resulting from the identifiable marker(s) is measured relative to the phenotype resulting from the reference marker. For example, if a collection of cells is produced such that each cell carries a single copy of a reference marker (e.g. monomeric red fluorescent protein; mRFP; Campbell RE et al., PNAS, 99:7877-7882, 2002 or mCherry; Shaner et al., Nat. Biotechnol. 22, 1567-72 (2004)), and a variable number of copies of the identifiable marker GFP, cells might then be sorted to enrich for those with high ratios of GFP intensity to RFP intensity. Simultaneous measurement of both genes can provide a phenotypic measure of gene dosage of the identifiable marker that is less subject to random variation between cells (e.g., in overall transcriptional or translational efficiency of a particular cell), as described previously in other contexts (Swain PS et al., “Intrinsic and extrinsic contributions to stochasticity in gene expression,” PNAS, 99(20):12795-12800, (2002); Elowitz MB et al. “Stochastic gene expression in a single cell,” Science 297(5584):1183-1186, (2002).

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

As used herein, the term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990), incorporated herein by reference in its entirety for all purposes. Regulatory sequences include those which direct expression of the nucleotide sequence encoding the identifiable marker only when induced (e.g., inducible promoters) or under certain conditions (e.g., conditional promoters), those which direct expression of the nucleotide sequence encoding the identifiable marker only in certain host cells (e.g., tissue-specific regulatory sequences), those which direct expression of the nucleotide sequence encoding the identifiable marker only at certain times (e.g., temporal-specific regulatory sequences) and those which direct constitutive expression of a nucleotide sequence encoding the identifiable marker in many types of host cells (e.g., cell type-specific or species-specific promoters). For reviews of eukaryotic transcription see Fickett et al. (1997) Genome Res. 7:861 and Barberis; and Transcription Activation in Eukaryotic Cells: Encyclopedia of Life Sciences, Macmillan Publishers Ltd., Nature Publishing Group (2003), incorporated herein by reference in their entirety for all purposes. In certain aspects, so as to facilitate correlation of signal intensity with identifiable marker gene copy number where a single identifiable marker is used, one or both of the identifiable marker gene and the regulatory sequence to which it is operably linked should be selected so that expression of the marker gene is relatively insensitive to the influence of sequences near which it might be inserted in the genome of the target cell or organism (i.e., such that its expression is robust with respect to positional effects).

Where the accumulation of copies of a single identifiable marker over successive rounds of insertion into the genome of the target cell or organism according to the invention results in signal saturation for the detection means employed, it may be useful to perform subsequent steps using either (a) a different identifiable marker, or (b) the same marker operably linked to a different regulatory element, such that expression of newly-inserted marker genes is induced, while expression of the earlier-inserted marker genes is not. Selection on the basis of multiple identifiable (i.e., continuously selectable) marker types could be carried out from the beginning of the process, replacing each identifiable marker selection step (such as in Example II), with a set of identifiable marker selections carried out in parallel according to option (a). Where multiple identifiable markers are used in parallel, and the variety of available markers has become exhausted, a set or group of markers can be re-used serially according to option (b).

The present invention can be used for a variety of organisms in which mating and recombination occurs. For example, methods of targeted gene replacement in C. elegans are known which could be used to produce single-mutant C. elegans strains (Barrett et al. (2004) Nat. Genet., 36:1231, incorporated herein by reference in its entirety for all purposes). Such strains can be mated with one another and progeny may be sorted by detecting an increase in identifiable markers as described further herein. For example, C. elegans can be sorted on the basis of the fluorescence activity of an identifiable marker (O'Connor, COPAS Application Note B-07, C. elegans Green Color Fluorescence Sorting website: unionbio/applications/app_notes/c_eleg_files/CelegansANB07.pdf (Union Biometrica, Somerville, Mass., USA), incorporated herein by reference in its entirety for all purposes).

Exogenous nucleic acid sequences can be targeted for delivery to target eukaryotic cells via conventional transformation or transfection techniques. In certain aspects, the introduced nucleic acid sequences and the target eukaryotic cells together are capable of targeted recombination. As used herein, the terms “transformation” and “transfection” are intended to refer to a variety of art-recognized techniques for introducing an exogenous nucleic acid sequence (e.g., DNA) into a target cell, including calcium phosphate or calcium chloride co-precipitation, DEAE-dextran-mediated transfection, lipofection, electroporation, optoporation, injection and the like. Suitable methods for transforming or transfecting target cells can be found in Sambrook et al. (Current Protocols in Molecular Biology, John Wiley & Sons, Inc., 1998, incorporated herein by reference in its entirety for all purposes), and other laboratory manuals. Methods of transfecting and transforming in the absence of targeted recombination can be useful for introducing a defined collection of nucleic acids (e.g., genes) into organisms in which targeted recombination is problematical.

For example, target cells can be cells such as yeast, C. elegans cells, insect cells, plant cells, Xenopus cells, or mammalian cells (such as Chinese hamster ovary cells (CHO), mouse cells, African green monkey kidney cells (COS), fetal human cells (293T) or other human cells). Other suitable target cells are known to those skilled in the art. Both cultured and explanted cells may be used according to the invention. The present invention is also adaptable for in vivo use using viral vectors including, but not limited to, replication defective retroviruses, adenoviruses, adeno-associated viruses and the like. For in vivo use, marker selection can be applied to the entire organism such as, for example, by using an automated worm sorter (Union Biometrica, Zurich Switzerland).

Target cells useful in the present invention include human cells including, but not limited to, embryonic cells, fetal cells, and adult stem cells. Human stem cells may be obtained, for example, from a variety of sources including embryos obtained through in vitro fertilization, from umbilical cord blood, from bone marrow and the like. In one aspect of the invention, target human cells are useful as donor-compatible cells for transplantation, e.g., via alteration of surface antigens of non-compatible third-party donor cells, or through the correction of genetic defect in cells obtained from the intended recipient patient. In another aspect of the invention, target cells, including without limitation human cells, are useful for the production of therapeutic proteins, peptides, antibodies and the like.

The target cells of the invention can also be used to produce nonhuman transgenic, knockout or other genetically-modified animals. Such animals include those in which a gene or nucleic acid is altered in part, e.g., by small or large deletions and/or insertions of target nucleic acid sequences. For example, in one embodiment, a target cell of the invention is a fertilized oocyte or an embryonic stem cell into which the addition or deletion of one or more nucleic acids has been performed. Such target cells can then be used to create non-human transgenic animals in which exogenous detectable translation product sequences have been introduced into their genome. As used herein, a “transgenic animal” is a non-human animal, such as a mammal, e.g., a rodent such as a guinea pig, rat, mouse or the like, in which one or more of the cells of the animal includes one or more exogenous genes. Other examples of transgenic animals include non-human primates, cows, goats, sheep, pigs, rabbits, ferrets, dogs, cats, chickens, amphibians, and the like. An exogenous gene is exogenous DNA that is integrated into the genome of a cell from which a transgenic animal develops and which remains in the genome of the mature animal. A knockout is the removal of endogenous DNA from a cell from which a knockout animal develops, which remains deleted from the genome of the mature animal. Methods for generating transgenic and knockout animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al., and in Hogan, B., Manipulating the Mouse Embryo, (Cold Spring Harbor Laboratory Press, Cold Spring Harbor, N.Y., 1986), incorporated herein by reference in their entirety for all purposes.

This invention is further illustrated by the following examples, which should not be construed as limiting. The contents of all references, patents and published patent applications cited throughout this application are hereby incorporated by reference in their entirety for all purposes.

### EXAMPLE I

Construction of a Collection of Single Deletion Pro Strains in S. Cerevisiae

The following approach may be used for introducing many deletions efficiently. The basic outline of the procedure is as follows. A collection of strains corresponding to the chosen gene set, each deleted for a single gene in that set, is generated. Into each deletion locus, a detectable marker (e.g., a green fluorescent protein gene (GFP)) is placed under the control of an inducible promoter (e.g., the Tet-promoter and the doxycycline-inducible activator rtTA (see Gossen et al. (1992) Proc. Natl. Acad. Sci. USA, 89:5547; and Urlinger et al. (2000) Proc. Natl. Acad. Sci. USA, 97:7963), incorporated herein by reference in their entirety for all purposes). In Example II, the collection of single-deletion GFP strains will be pooled, and the pool will be taken through repeated rounds of mating and sporulation. At each round, those cells carrying the most deletions will be selected by detecting increases in marker activity. For example, cells carrying many deletions may be selected on the basis of increased GFP protein activity caused by higher GFP gene dosage. Selection may be achieved, for example, using fluorescence activated cell sorting (FACS).

Stage 1: The Starter Strains

Plasmid 1: MARKER1, a marker for selecting the presence of the plasmid; MARKERa for selecting a haploids (e.g., STE2pr-HIS3); and MARKERα for selecting α haploids (STE3pr-LEU2).

Targeting Construct 1: MARKER2 and CMVpr-rtTA (a constitutively expressed doxycycline-dependent activator of tetO promoters) will be generated with a flanking sequence designed to replace CAN1 by homologous recombination.

Targeting Construct 2: MARKER3 and CMVpr-rtTA will be generated with a flanking sequence designed to replace CAN1 by homologous recombination.

Targeting Construct 3: MARKER4 TetO-GFP construct will be generated with a flanking sequence designed to replace kanMX by homologous recombination.

A collection of strains, each carrying a single deletion locus, wherein each locus is replaced with a selectable marker (e.g., GFP) under control of an inducible promoter (e.g., the tetO promoter and the doxycycline-inducible activator rtTA), can be generated using the protocol set forth above.

Methods

Constructing a or α Starter Strains

Step 1: Plasmid 1 will be introduced into the α starter strain and transformants for Leu⁺ and/or MARKER1 will be selected.

Step 2: Gene replacement of CAN1 will be directed in the yeast chromosome with MARKER2 using Targeting Construct 1. Transformants will be selected for Marker1⁺ and counter-selected against Can1⁺ with canavanine.

Constructing a or α Deletion Strains from an Existing Haploid KanMX Deletion Strains for a Nucleic Acid Sequence of Interest

Step 1: An α-haploid S288C strain and will have one copy of kanMX replaced with MARKER4 linked to TetO-GFP using Targeting Construct 3, and will be selected for Marker4⁺.

The Saccharomyces Genome Project has revealed the presence of more than 6000 open reading frames (ORFs) in the S. cerevisiae genome. Nearly all ORFs larger than 100 codons have been disrupted and replaced with a KanMX module, and uniquely tagged with one or two 20mer sequence(s) (Saccharomyces Genome Deletion Project web page, Stanford University: http://sequence.stanford.edu/group/yeast_deletion_project/deletions3.html, incorporated herein by reference in its entirety for all purposes or http://www.openbiosystems.com/Genomics/Model%20Organism%20Resources/Yeast%20Resources%Yeast%20Knockout%20Strains/ incorporated herein by reference in its entirety for all purposes). Accordingly, a variety of yeast strains are available for use in step 1.

Step 2: CAN1 will be replaced in the chromosome with MARKER3 using Targeting Construct 2. Transformants will be selected for Marker3⁺ and counter-selected for Can1⁺ with canavanine. The resulting α deletion strains will then be checked for proper GFP integration by performing deletion-strain-specific PCR. The resulting α deletion strains should be KanS.

Step 3: The α deletion strain will be mated with the a starter strain and expression of the four MARKER genes (i.e., MARKER1, MARKER2, MARKER3, MARKER4) will be selected. This will generate an “a/α PrePro” strain. The a/α PrePro strain will be checked for GFP activity induced by doxycycline and for His⁻ and Leu⁻.

Step 4: First and second cultures of the a/α PrePro strain are sporulated.

Step 5a: The first culture will be selected for His⁺, MARKER1, MARKER2, MARKER4 a-haploids to yield an “a Pro” strain. The a Pro strain should be KanS, Marker3⁻, CanR, and Leu⁻.

Step 5b: The second culture will be selected for Leu⁺, MARKER1, MARKER3, MARKER4 α-haploids to yield an “α Pro” strain. The α Pro strain should be KanS, Marker2⁻, CanR, and His⁻.

A variety of yeast strains and plasmids that can be used in the present invention are known in the art (see e.g., Baudin et al. (1993) Nucleic Acids Res. 21:3329; Wach et al. (1994) Yeast 10:1793, incorporated herein by reference in their entirety for all purposes).

### EXAMPLE II

### Generating Strains Deleted for a Desired Set of Genes in S. Cerevisiae

Step 1: Desired single mutant strains are pooled generating:

1) A pooled collection of a Pro strains corresponding to genes that are desired to be deleted. As used herein, this pooled collection is referred to as the H0a pool (Haploid 0^{th} round a-type).

2) A pooled collection of α Pro strains corresponding to genes that are desired to be deleted. As used herein, this pooled collection is referred to as the H0α pool (Haploid 0^{th} round α-type).

Step 2: H0a pools and H0α pools are mated, and diploids are selected by identifying cells that are Marker2+ and Marker3+. As used herein, this mated pool is referred to as the D0 pool (Diploid 0^{th} round a/α).

Step 3: D0 will be sporulated and one of the following will be selected for:

1) His⁺, MARKER2 a haploids (i.e., the H1a pool for Haploid 0^{th} round a-type).

2) Leu⁺, MARKER3 α haploids (i.e., the H1α pool for Haploid 0^{th} round α-type).

Step 4: The cells from each of the H1 pools from step 3 will be sorted to select for marker expression (e.g., GFP fluorescence) and haploid (1N) DNA content. Strains carrying two deletions should represent approximately 25% of the H1 population if the corresponding genes are unlinked. If cells are selected from the goth percentile for marker expression (e.g., GFP activity), the resulting selected H1 pools should only contain cells carrying two deletions. If the two deletions are genetically linked (e.g., proximal on the same chromosome), doubly-deleted strains may represent a smaller fraction of the population.

Step 5: Steps 2-4 will be repeated, mating H1a and H1α pools to generate an H2a/α population. Strains carrying four deletions should represent only about 6% of the H2a/α population if the corresponding genes are unlinked. If cells are selected from the 95^{th} percentile for marker expression (e.g., GFP activity), the resulting H2 population should only contain cells carrying four or more deletions.

Step 6: Steps 2-4 will be repeated, mating H2a and H2α pools to generate an H3a/α population. Strains carrying eight deletions should represent only 0.4% of the H3a/α population, and strains carrying seven or more deletions will represent 3.5% of the population if the corresponding genes are unlinked. If cells are selected from the 99^{th} percentile for marker expression (e.g., GFP activity), the resulting H3 cells will carry seven or more deletions.

Step 7: Steps 2-4 will be repeated mating H3a and H3α pools, H4a and H4a pools, H5a and H5α pools, H6a and H6α pools, and so on, to generate H4, H5, H6, and H7 pools, respectively. Using cumulative binomial distribution, assuming the progeny to have a 50% chance of receiving any given marker (e.g., GFP) allele and assuming the markers are independently assorted (i.e., unlinked), the following numbers of deletions will be generated: H4 pools will contain cells carrying 11 or more deletions; H5 pools will contain cells carrying 16 or more deletions; H6 pools will contain cells carrying 23 or more deletions; H7 pools will contain cells carrying 31 or more deletions; H8 pools will contain cells carrying 40 or more deletions; H9 pools will contain cells carrying 55 or more deletions; H13 pools will contain cells carrying 67 or more deletions; H11 pools will contain cells carrying 89 or more deletions; H12 pools will contain cells carrying 108 or more deletions; and H13 pools will contain cells carrying 129 or more deletions. The largest number of genes N for which more than 1% of the population has N or more genes is listed above.

The number of deletions obtained are subject to fluctuations inherent to any stochastic process. These calculations are based on the assumption that the number of cells carried through the process is sufficient to avoid fixation of wild type alleles, i.e., loss of all cells carrying a deletion allele at a particular locus. If some pairs of genes among those genes to be deleted are linked, deletions may be acquired more slowly, but the same principles will hold. These calculations are also premised on the assumption that there is no fitness effect to the deletion of any gene involved. Fitness effects may cause deletions to be acquired more gradually within this process.

A variation of the method described above may be used when an allele is fixed. The entire process can be carried out in parallel, to generate many H0a and H0α pools, H1a and H1α pools, H2a and H2α pools, and the like. Instead of mating H2a and H2α pools produced from the same progenitor H1a and H1α pools, one may, for example, mate an H2a pool with an H2α pool that did not derive from the same H1a and H1α pools. Such “outbreeding” or mating between “geographically isolated” pools will reduce the chances that the wild type allele becomes fixed, since the same allele is unlikely to have become fixed in two independently-generated strain pools.

Characterization of Strains

Barcode assays may be used to detect deletions present. As used herein, the term “barcode” refers to a unique DNA sequence that can be used to flank one or both ends of each deletion in an organism, e.g., yeast. The unique sequence serves as a molecular “barcode” that allow yeast strains to be identified. Barcode assays are particularly useful for determining the genetic basis of drug sensitivity and resistance. Bar code technologies are known in the art (see Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240; incorporated herein by reference in their entirety for all purposes).

Heightened marker activity may in some cases result from aneuploidy, i.e., atypical copy number in whole chromosomes or chromosomal regions. For this reason, strains resulting from the above process should be screened to remove those with aberrant copy number. Genome-scale methods for quantifying copy number are known in the art (Pollack et al. (1999) Nat. Genet. 23:41; Pinkel et al. (1998) Nat. Genet. 20:207; Bond et al. (2004) Curr. Genet. 45:360; Infante et al. (2003) Genetics 165:1745; incorporated herein by reference in their entirety for all purposes).

### EXAMPLE III

### References

Each of the following is incorporated herein by reference in its entirety for all purposes:
Huang et al. (2003) Proc. Natl. Acad. Sci. USA 100:11529
Becskei et al. (2001) Embo J. 20:2528
Hutchison et al. (1999) Science 286:2165
Westers et al. (2003) Mol. Biol. Evol. 20:2076
Mellado et al. (1998) Extremophiles 2:435
Islas et al. (2004) Orig. Life Evol. Biosph. 34:243
Koonin. (2003) Nat. Rev. Microbiol. 1:127

### EQUIVALENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

What is claimed is:

1. A method for altering a nucleic acid sequence in a eukaryotic cell comprising the steps of:
generating an alteration in a nucleic acid sequence in a first eukaryotic cell, wherein the alteration is accompanied by a first identifiable marker;
generating an alteration in a nucleic acid sequence in a second eukaryotic cell, wherein the alteration is accompanied by a second identifiable marker;
combining the first and second eukaryotic cells to generate a resulting eukaryotic cell that includes the first identifiable marker and the second identifiable marker, wherein the number of markers in the resulting eukaryotic cell is lower than the number of alterations in the resulting eukaryotic cell;
optionally detecting one or more of the first identifiable marker and the second identifiable marker in the resulting eukaryotic cell; and
optionally using one or more of the first identifiable marker and the second identifiable marker to select a resulting eukaryotic cell having a plurality of alterations.

2. The method of claim 1, wherein the resulting eukaryotic cell is generated by mating and sporulation or by allowing homologous recombination between the nucleic acid sequence in the first eukaryotic cell and the nucleic acid sequence in the second eukaryotic cell to produce in the resulting eukaryotic cell a single nucleic acid sequence including the first identifiable marker and the second identifiable marker.

3. The method of claim 1, further including the step of successively combining a resulting eukaryotic cell with a eukaryotic cell including one or more identifiable markers until a desired eukaryotic cell having a plurality of identifiable markers is generated, and optionally using one or more of the first identifiable marker and the second identifiable marker to select a desired eukaryotic cell having a plurality of alterations.

4. The method of claim 3, wherein the step of using one or more of the first identifiable marker and the second identifiable marker to select a desired eukaryotic cell includes detecting an increased copy number of the marker.

5. The method of claim 3, wherein the desired eukaryotic cell is generated by allowing homologous recombination between a nucleic acid sequence in the resulting eukaryotic cell and a nucleic acid sequence in the eukaryotic cell including one or more identifiable markers to produce the desired eukaryotic cell having a single nucleic acid sequence including the plurality of identifiable markers.

6. The method of claim 1, wherein the steps of generating an alteration in the first and second eukaryotic cells include deleting a nucleic acid sequence.

7. The method of claim 1, wherein the steps of generating an alteration in the first and second eukaryotic cells include adding a nucleic acid sequence.

8. The method of claim 1, wherein the steps of generating an alteration in the first and second eukaryotic cells include substituting an endogenous nucleic acid sequence with an exogenous nucleic acid sequence.

9. The method of claim 1, wherein the steps of generating an alteration in the first and second eukaryotic cells include deleting a plurality of nucleic acid sequences.

10. The method of claim 1, wherein the steps of generating an alteration in the first and second eukaryotic cells include adding a plurality of nucleic acid sequences.

11. The method of claim 1, wherein the steps of generating an alteration in the first and second eukaryotic cells include substituting each of a plurality of endogenous nucleic acid sequences with a corresponding exogenous nucleic acid sequence.

12. The method of claim 1, wherein the first identifiable marker and the second identifiable marker are the same identifiable marker.

13. The method of claim 1, wherein the first identifiable marker and the second identifiable marker are separately identifiable.

14. The method of claim 1, wherein the first identifiable marker and the second identifiable marker each includes a regulatory sequence.

15. The method of claim 1, wherein the steps of generating an alteration in the first and second eukaryotic cells include one or more of deleting a nucleic acid sequence, adding a nucleic acid sequence and substituting a nucleic acid sequence.

16. The method of claim 1, further including the step of inserting a reference marker in the first eukaryotic cell and inserting a reference marker in the second eukaryotic cell, wherein each of the reference markers is separately identifiable from the first or second identifiable markers.

17. The method of claim 3, wherein the desired eukaryotic cell contains a minimal eukaryotic genome.

18. The method of claim 3, wherein the desired eukaryotic cell contains genes that are either deleted from or inserted into one or more selected cellular pathways.

19. The method of claim 3, wherein all genes that encode components of a cellular pathway in the desired eukaryotic cell have been deleted.

20. The method of claim 3, wherein all genes that encode components of a cellular pathway in the desired eukaryotic cell have been deleted and replaced with genes for a different cellular pathway.

21. The method of claim 3, wherein one or more markers in the resulting eukaryotic cell are removed or replaced with different markers.

22. A method for altering a nucleic acid sequence in a eukaryotic cell comprising the steps of:
generating a deletion in a nucleic acid sequence in a first eukaryotic cell, wherein the deletion is accompanied by a first identifiable marker;
generating a deletion in a nucleic acid sequence in a second eukaryotic cell, wherein the deletion is accompanied by a second identifiable marker;
combining the first and second eukaryotic cells to generate a resulting eukaryotic cell that includes the first identifiable marker and the second identifiable marker, wherein the number of markers in the resulting eukaryotic cell is lower than the number of deletions in the resulting eukaryotic cell;
optionally detecting one or more of the first identifiable marker or the second identifiable marker in the resulting eukaryotic cell; and
optionally using one or more of the first identifiable marker and the second identifiable marker to select a resulting eukaryotic cell having a plurality of deletions.

23. The method of claim 22, wherein the step of using one or more of the first identifiable marker and the second identifiable marker to select a resulting eukaryotic cell includes detecting an increased copy number of the marker.

24. A method for altering a nucleic acid sequence in a eukaryotic cell comprising the steps of:
generating an insertion in a nucleic acid sequence in a first eukaryotic cell, wherein the insertion is accompanied by a first identifiable marker;
generating an insertion in a nucleic acid sequence in a second eukaryotic cell, wherein the insertion is accompanied by a second identifiable marker;
combining the first and second eukaryotic cells to generate a resulting cell that includes the first identifiable marker and the second identifiable marker, wherein the number of markers in the resulting eukaryotic cell is lower than the number of insertions in the resulting eukaryotic cell;
optionally detecting one or more of the first identifiable marker and the second identifiable marker in the resulting eukaryotic cell; and
optionally using one or more of the first identifiable marker and the second identifiable marker to select a resulting eukaryotic cell having a plurality of insertions.

25. The method of claim 24, wherein the step of using one or more of the first identifiable marker and the second identifiable marker to select a resulting eukaryotic cell includes detecting an increased copy number of the marker.

26. A method for generating a eukaryotic cell having a plurality of altered nucleic acid sequences and associated markers comprising the steps of:
performing successive homologous recombination of altered nucleic acid sequences including identifiable markers between eukaryotic cells until a desired eukaryotic cell is produced having alterations selected from the group consisting of a plurality of identifiable markers, a plurality of inserted genes and a plurality of deleted genes, wherein the number of markers in the desired eukaryotic cell is lower than the number of alterations in the desired eukaryotic cell; and
optionally using one or more of the plurality of identifiable markers to select a desired eukaryotic cell having a plurality of alterations.

27. The method of claim 26, wherein the step of using one or more of the plurality of identifiable markers to select a desired eukaryotic cell includes detecting an increased copy number of the marker.
